(19) United States
(12) Patent Application Publication
(10) Pub. No.: US 2003/0232048 A1
(43) Pub. Date: Dec. 18, 2003

(54) Methods for treating chronic obstructive pulmonary disease (COPD)

(76) Inventors: Xiao-dong Yang, Palo Alto, CA (US); Gregory Bell, Tiburon, CA (US); Ronald P. Gladue, Stonington, CT (US); Elizabeth M. Kudlacz, Groton, CT (US); Henry J. Showell, Westbrook, CT (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/392,635

(22) Filed: Mar. 18, 2003

Related U.S. Application Data
(60) Provisional application No. 60/366,426, filed on Mar. 19, 2002.

(51) Int. Cl. A61K 39/395

(52) U.S. Cl. 424/141.1; 424/145.1

(57) ABSTRACT

The present disclosure relates to methods of treating Chronic Obstructive Pulmonary Disease (COPD) and its various indications, particularly including chronic bronchitis, emphysema, and irreversible asthma. Treatment regimens generally include the administration of anti-interleukin-8 antibodies to the patient to reduce the severity of an inflammatory response by the patient's immune system.

### RELATED APPLICATIONS

This application claims priority to, and hereby incorporates by reference in its entirety, provisional U.S. Patent Application No. 60/366,426, filed Mar. 19, 2002, and entitled “METHODS FOR TREATING CHRONIC OBSTRUCTIVE PULMONARY DISEASE (COPD).”

### BACKGROUND OF THE INVENTION

1. Field of the Invention

Anti-interleukin-8 antibodies are described for use in the treatment of chronic obstructive pulmonary disease (COPD).

2. Description of the Related Art

Chronic Obstructive Pulmonary Disease (COPD) is one of the most common chronic conditions and the fourth leading cause of death in the United States. COPD includes several related disorders that restrict the patient's ability to exhale. Accordingly, patients frequently experience dyspnea, or shortness of breath. Dyspnea typically causes patient discomfort, limits the patient's ability to engage in physical activity, and can induce further adverse health effects due to a diminished supply of oxygen. The two most common disorders associated with COPD are chronic bronchitis and emphysema, though patients suffering from COPD may also have chronic asthma, bronchiectasis, immunoglobulin deficiency, and cystic fibrosis.

Although various environmental toxins are believed to contribute to COPD, cigarette smoking is the most common cause. Cigarette smoke is believed to be the cause of more than 80% of all COPD cases. Cigarette smoke contains harmful irritants that inflame the airways and the lungs. In turn, this inflammation triggers a series of biochemical events in the body's immune system which cause substantial damage of the lungs and airways.

This immune response occurs when macrophages and endothelial cells in the inflamed tissue secrete the protein interleukin-8 (IL-8), a chemotactic factor which attracts and activates neutrophils (phagocytic cells which respond to the inflammation.) These neutrophils leave the blood stream and are drawn toward the high IL-8 concentration. Upon reaching the site of inflammation, the activated neutrophils produce and release the infection-fighting enzyme neutrophil elastase. Unfortunately, in a massive neutrophil response, the production and secretion of neutrophil elastase overwhelms the tissue and breaks down the elastic and structural elements in the lung parenchyma leading to lung and airway damages. This irreversible damage to the lung causes the initial shortness of breath which is common in most COPD patients. As the condition progresses, the lung capacity decreases further and patients may experience coughing, wheezing, increased mucous production, and infection. As the lung capacity decreases, poor ventilation reduces oxygen levels (hypoxia) and increases carbon dioxide levels (hypercapnia) in the body. Patients with prolonged and severe hypoxia and hypercapnia risk respiratory failure, heart rhythm abnormalities, and other life threatening conditions.

IL-8 is a member of the C-X-C chemokine family and acts as the primary chemoattractant for neutrophils implicated in many inflammatory diseases, including ARDS, rheumatoid arthritis, inflammatory bowel disease, glomerlonephritis, psoriasis, alcoholic hepatitis, reperfusion injury, to name a few. Moreover, IL-8 is a potent angiogenic factor for endothelial cells and has been implicated in tumor angiogenesis

Others have described anti-IL-8 antibody technologies which have been developed and disclosed for treating bacterial pneumonia (U.S. Pat. No. 5,686,070), asthma (U.S. Pat. No. 5,874,080), and ulcerative colitis (U.S. Pat. No. 5,707,622).

What is needed in the art is a safe and effective treatment for COPD and its various indications, including, for example, chronic bronchitis, emphysema, and irreversible asthma.

### SUMMARY OF THE INVENTION

One aspect of the invention is a method of treating a patient suffering from symptoms of Chronic Obstructive Pulmonary Disease (COPD) including administering an amount of an antibody specific for human interleukin-8 (IL-8) effective to reduce the symptoms. In preferred embodiments, the antibody is capable of neutralizing or down-regulating the activity of IL-8 in the patient. Preferred antibody delivery routes include intravenous, intraperitoneal, inhalation, intramuscular, subcutaneous, and oral administration. Preferably, the antibody is a monoclonal antibody. More preferably, the antibody is a fully human antibody, such as an ABX-IL8 antibody, available from Abgenix, Inc. (Fremont, Calif.).

Another aspect of the present invention is a method of treating the various indications of COPD, including chronic bronchitis, emphysema, and irreversible asthma. In particular, one aspect of the present invention is a method of treating a patient suffering from symptoms of chronic bronchitis including administering an amount of an antibody specific for human IL-8 effective to reduce the symptoms. In preferred embodiments, the antibody is capable of neutralizing or down-regulating the activity of IL-8 in the patient. Preferred antibody delivery routes include intravenous, intraperitoneal, inhalation, intramuscular, subcutaneous, and oral administration. Preferably, the antibody is a monoclonal antibody. More preferably, the antibody is a fully human antibody, such as an ABX-IL8 antibody.

In a further aspect of the invention, anti-IL-8 antibodies can be formulated in a pharmaceutically acceptable vehicle which is then administered to a patient suffering from COPD or any of its indications.

### BRIEF DESCRIPTION OF THE DRAWINGS

FIG. 1 shows neutrophil chemotaxis as a function of ABX-IL8 concentration.

FIG. 2 shows inhibition percentage of neutrophil chemotaxis by ABX-IL8 in an experiment using COPD sputum at a 1:10 dilution.

FIG. 3 shows inhibition percentage of neutrophil chemotaxis by ABX-IL8 in an experiment using COPD sputum at a 1:100 dilution.

FIG. 4 shows the amount of IL-8 detected in the sputum of COPD patients.

FIG. 5 shows the inhibition of IL-8 induced neutrophil activation by ABX-IL8 in a rat study.

FIG. 6 shows neutrophil quantity in rats given various amounts of human IL-8.

FIG. 7 shows neutrophil quantity in rats given human IL-8 and ABX-IL-8.

### DETAILED DESCRIPTION OF THE PREFERRED EMBODIMENTS

One embodiment of the invention is a method for treating inflammatory diseases of the lung by administration of an antibody capable of binding to interleukin-8 (IL-8). For example, chronic obstructive pulmonary disease (COPD), can be treated by administration to a patient of an anti-IL-8 antibody. COPD can include several indications relating to inflammation of the lungs and respiratory tract, such as chronic bronchitis, emphysema, and irreversible asthma. These indications have common features, including in particular, dyspnea or shortness of breath caused by damage to the respiratory tract. Hence, it is expected that anti-IL-8 antibodies can be used to treat any indication of COPD.

In one embodiment, a patient suffering from COPD is given intravenous or oral dosages of anti-IL-8 antibodies in a pharmaceutically acceptable vehicle. This treatment is effective to reduce the symptoms of COPD in the patient. In one embodiment, 0.1-10 mg/kg body weight of anti-IL-8 antibodies are administered to the patient. More preferably, 1-10 mg/kg body weight of anti-IL-8 antibodies are administered. Preferably, this dosage is repeated each month as needed. Alternative dosages and dose schedules are discussed infra.

Definitions:

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures arc generally performed according to conventional methods well known in the art and as described in various general and more specific references that are cited and discussed throughout the present specification. See e.g. Singleton et al., Dictionary of Microbiology and Molecular Biology 2^{nd} ed., J. Wiley & Sons (New York, N.Y. 1994); Sambrook et al. Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

“COPD” refers to chronic obstructive pulmonary disorder and/or any of its indications, including for example, chronic bronchitis, emphysema, irreversible asthma, bronchiectasis, immunoglobulin deficiency, and cystic fibrosis. Hence, a reference to “treating COPD in a patient,” is intended to include, for example, “treating chronic bronchitis in a patient,” assuming that the patient in question has chronic bronchitis.

“Polymerase chain reaction” or “PCR” refers to a procedure or technique in which minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol. 51:263 (1987); Erlich, ed., PCR Technology (Stockton Pres, NY, 1989). A used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al. J. Mol. Biol. 186:651 (1985; Novotny and Haber, Proc. Natl. Acad. Sci. U.S.A. 82:4592 (1985); Chothia et al., Nature 342:877-883 (1989)).

The term “antibody” herein is used in the broadest sense and specifically covers intact monoclonal antibodies, polyclonal antibodies, multispecific antibodies (e.g. bispecific antibodies) formed from at least two intact antibodies, and antibody fragments including Fab and F(ab)′2, so long as they exhibit the desired biological activity. The “light chains” of antibodies (immunoglobulins) from any vertebrate species can be assigned to one of two clearly distinct types, called K and λ, based on the amino acid sequences of their constant domains.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature, 352:624-628 (1991) and Marks et al., J. Mol. Biol., 222:581-597 (1991), for example.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

A “neutralizing antibody” is an antibody molecule which is able to eliminate or significantly reduce an effector function of a target antigen to which is binds. Accordingly, a “neutralizing” IL-8 antibody is capable of eliminating or significantly reducing an effector function, such as IL-8 activity.

“Antibody-dependent cell-mediated cytotoxicity” and “ADCC” refer to a cell-mediated reaction in which non-specific cytotoxic cells that express Ig Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcyRIII only, whereas monocytes express FcyRI, FcyRII and FcyRIII. FcRs expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362, or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1988).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the Ig light-chain and heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al. (1991). The constant domains are not involved directly in binding an antibody to an antigen, but exhibit various effector functions, such as participation of the antibody in antibody-dependent cellular toxicity.

“Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-62 (L2), and 89-97 (L3) in the light chain variable domain and 31-55 (H1), 50-65 (H2) and 95-102 (H3) in the heavy chain variable domain; Kabat et al., Sequences of Proteins of Immunological Interest, 5^{th} Ed. Public Health Service, National Institutes of Health, Bethesda, Md. (1991)) and/or those residues from a “hypervariable loop” (e.g. residues 26-32 (L1, 50-52 (L2) and 91-96 (L3) in the light chain variable domain and 26-32 ((H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

The term “complementarity determining regions” or “CDRs” when used herein refers to parts of immunological receptors that make contact with a specific ligand and determine its specificity. The CDRs of immunological receptors are the most variable part of the receptor protein, giving receptors their diversity, and are carried on six loops at the distal end of the receptor's variable domains, three loops coming from each of the two variable domains of the receptor.

The term “epitope” is used to refer to binding sites for (monoclonal or polyclonal) antibodies on protein antigens.

The term “amino acid” or “amino acid residue,” as used herein, refers to naturally occurring L amino acids or to D amino acids as described further below with respect to variants. The commonly used one and three-letter abbreviations for amino acids are used herein (Bruce Alberts et al, Molecular Biology of the Cell, Garland Publishing, Inc., New York (3d ed. 1994)).

The term “ABX-IL8 antibody” means an embodiment of a human anti-1L-8 antibody developed by Abgenix, Inc. of Fremont, Calif. (www.abgenix.com).

The term “disease state” refers to a physiological state of a cell or of a whole mammal in which an interruption, cessation, or disorder of cellular or body functions, systems, or organs has occurred.

The term “symptom” means any physical or observable manifestation of a disorder, whether it is generally characteristic of that disorder or not. The term “symptoms” can mean all such manifestations or any subset thereof.

The term “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

“Administer,” for purposes of treatment, means to deliver to a patient. Such delivery can be intravenous, intraperitoneal, by inhalation, intramuscular, subcutaneous, oral, topical, transdermal, or surgical.

“Therapeutically effective amount,” for purposes of treatment, means an amount such that an observable change in the patient's condition and/or symptoms could result from its administration, either alone or in combination with other treatment.

A “pharmaceutically acceptable vehicle,” for the purposes of treatment, is a physical embodiment that can be administered to a patient. Pharmaceutically acceptable vehicles can be, but are not limited to, pills, capsules, caplets, tablets, orally administered fluids, injectable fluids, sprays, aerosols, lozenges, neutraceuticals, creams, lotions, oils, solutions, pastes, powders, vapors, or liquids. One example of a pharmaceutially acceptable vehicle is a buffered isotonic solution, such as phosphate buffered saline (PBS).

“Neutralize,” for purposes of treatment, means to partially or completely suppress chemical and/or biological activity.

“Down-regulate,” for purposes of treatment, means to lower the level of a particular target composition.

“Mammal” for purposes of treatment refers to any animal classified as a mammal, including humans, domestic and farm animals, and zoo, sports, or pet animals, such as monkeys, dogs, horses, cats, cows, etc.

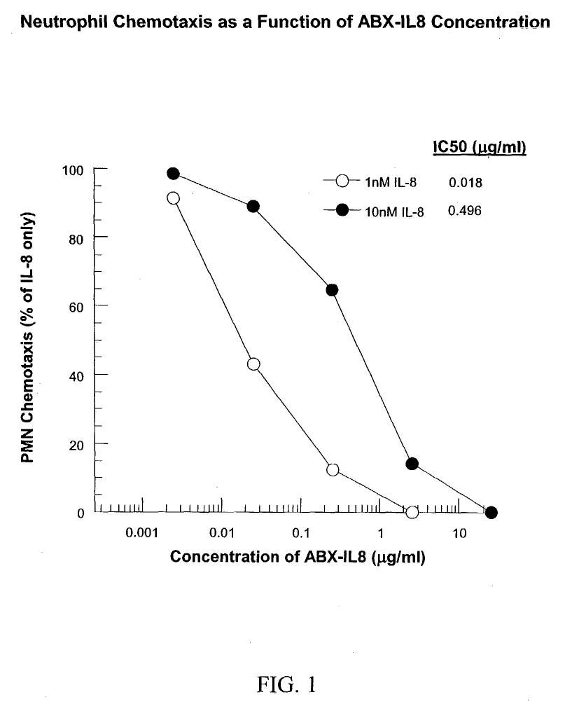

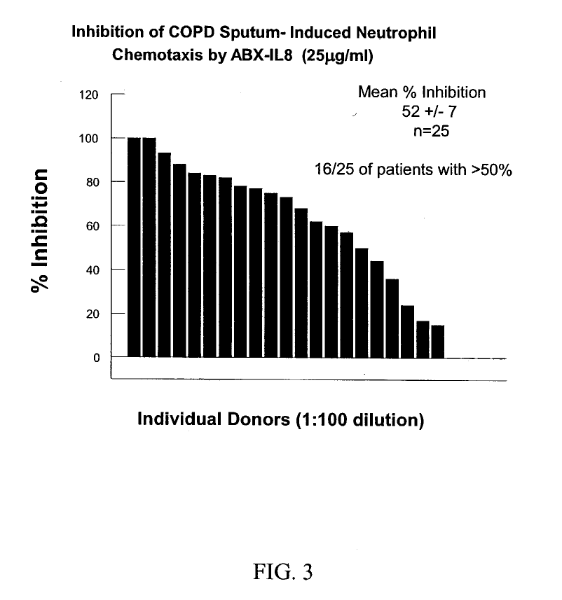

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules represented by FIGS. 1, 5, 9, 13, 17, 21, 25, and 29 and the human kappa light chain immunoglobulin molecules represented by FIGS. 3, 7, 11, 15, 19, 23, 27, and 31, as well as antibody molecules formed by combinations comprising the heavy chain immunoglobulin molecules with light chain immunoglobulin molecules, such as the kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as alpha.-, .alpha.-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physiocochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

In the present invention, anti-IL-8 antibodies can be administered to a patient suffering from COPD to improve the patient's condition. Accordingly, patients suffering from one or more of the various indications of COPD, such as chronic bronchitis, emphysema, irreversible asthma, bronchiectasis, immunoglobulin deficiency, and cystic fibrosis can be treated using anti-IL-8 antibodies according to the present invention.

In accordance with the present invention, anti-IL-8 antibodies can be administered to alleviate a patient's symptoms, or can be administered to counteract a mechanism of the disorder itself. It will be appreciated by those of skill in the art that these treatment purposes are often related and that treatments can be tailored for particular patients based on various factors. These factors can include the age, gender, or health of the patient, the progression of COPD, the degree of dyspnea, the amount of tissue damage to the patient's respiratory tract, the patient's smoking history, and various environmental factors (including, for example, temperature, humidity, and air pollution) which could contribute to the patient's condition. The treatment methodology for a patient can be tailored accordingly dosage, timing of administration, route of administration, and by concurrent or sequential administration of other therapies.

Example 8 infra describes one embodiment of the invention in which anti-IL-8 antibodies are administered to patients in an 800 mg loading dose followed by 400 mg doses administered monthly for three months. It is expected, however, that alternative dosages (particularly increased dosages) and alternative dosing schedules will also be effective. For example, a patient can be given approximately 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, 3000 mg, 3500 mg, 4000 mg, 4500 mg, or 5000 mg of anti-IL-8 antibodies or more per month. Further, a dosage of anti-IL-8 antibodies can be administered daily, semi-weekly, weekly, bi-weekly, monthly, bi-monthly, or on some other schedule that is convenient for the patient and any healthcare provider(s), and which allows pharmaceutical efficacy. Similarly, anti-IL-8 antibodies can be administered on demand according to the patient's present signs and/or symptoms, or upon exposure to exacerbating conditions, such as the presence of cigarette smoke. Considerations in selecting dosages and dosing schedules can include the patient's respiratory condition, age, body weight, sex, and the results of previous treatments.

Finally, it is contemplated that anti-IL-8 antibodies will be useful for treating other conditions in which IL-8 acts as a chemoattractant for inflammation, or otherwise mediates an adverse or destructive response. In addition to COPD, such conditions can include ARDS, rheumatoid arthritis, inflammatory bowel disease, glomerlonephritis, psoriasis, alcoholic hepatitis, reperfusion injury, tumor angiogenesis, and others.

### EXAMPLES

### Example 1

### Inhibition of Neutrophil Chemotactic Activity in Sputums of COPD Patients by anti-IL-8 Antibodies

Methods:

A total of 28 sputum samples from patients were obtained. Neutrophils were isolated from the peripheral blood of normal donors using previously established methods. See Ferrante, A. & Thong, Y.H. A Rapid One-step Procedure for Purification of Mononuclear and Polymorphonuclear Leukocytes from Human Blood Using a Modification of the Hypaque-Ficoll Technique, J. Immunol. Methods 24:389-393 (1978). Briefly, blood was collected in heparin and layered over Ficoll. Neutrophils were isolated and washed four times prior to use. Cells were resuspended at 4×10⁶/ml in RPMI medium containing 0.5% bovine serum albumin.

The chemotactic activity of neutrophils in the sputum was determined using a Boyden chamber. Two dilutions of sputum (1:10 and 1:100) were utilized and placed (in triplicate) into the lower chambers. A 50 μL suspension of 4×10⁶ neutrophils/ml was placed into thc upper chambers. Each dilution of sputum was tested alone and in the presence of 25 μg/ml of the human anti-IL-8 monoclonal antibody, ABX-IL8 (Abgenix, Inc., Fremont, Calif.), an amount previously determined to neutralize >90% of the chemotactic activity generated with 10 nM IL-8. A polycarbonate filter with 5 μl pore size separated the chambers.

After 45 minutes at 37° C., non-migrating cells from the upper surface of the filter were removed by scraping and the underside of the filter stained with Diff-Quik® stain. The number of migrating cells were counted by light microscopy from a minimum of 6 high power fields. Absolute migration was determined by subtracting out any random migration observed from those wells not containing any sputum.

Results:

FIG. 1 is a graph showing neutrophil chemotaxis as a function of the concentration of ABX-IL8 (measured in μg/mL) for the two concentrations of IL-8, 1 nM and 10 nM. As shown in FIG. 1, the amount of ABX-IL8 sufficient to neutralize more than 90% of the neutrophil chemotactic activity observed with a 10 nM concentration of recombinant IL-8 was 25 μg/ml. As such, this concentration was chosen in all experiments to assess the role of IL-8 on the neutrophil chemotactic activity from sputum samples taken from COPD patients.

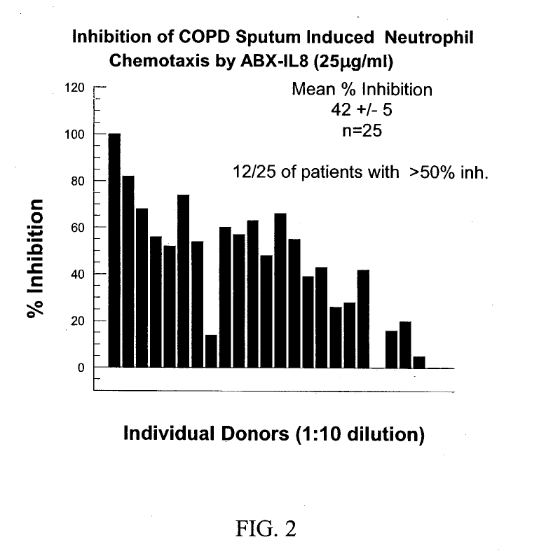

FIGS. 2 and 3 illustrate the inhibitory effects of ABX-IL8 on sputum-induced neutrophil chemotaxis at the 1:10 and 1:100 dilution, respectively. In both of these bar graphs, the percent inhibition in the range of 0 to 100% is shown for each of the individual donors appearing along the X-axis. The percent inhibition of chemotaxis was assessed using the mean migration from triplicate wells with and without 25 μg/ml of ABX-IL8. As shown in these figures, 12 out of the 25 patients exhibited greater than 50% inhibition in the 1:10 dilution and 16 out of 25 exhibited greater than 50% inhibition in the 1:100 dilution. Actual percent inhibition of each sputum specimen and the associated chemotactic index (CI, amount of chemotaxis observed relative to background) along with individual case history data are shown in Table 1.

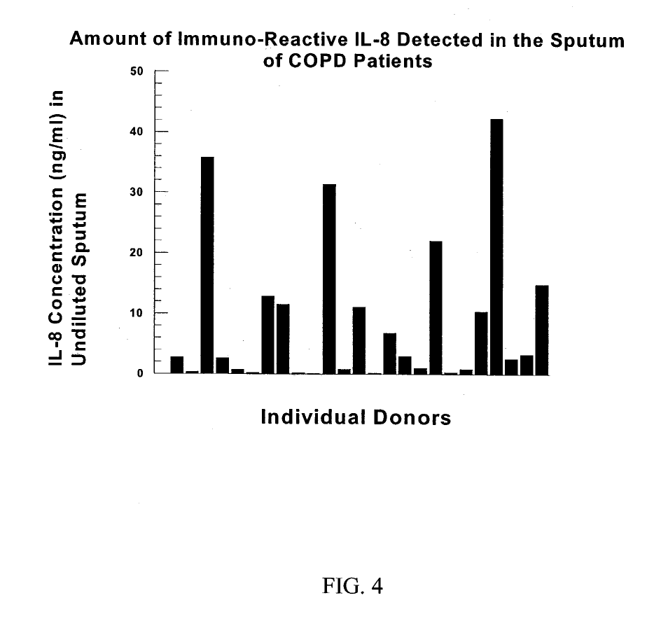

FIG. 4 illustrates the amount of immunoreactive IL-8 in each sputum sample as determined by ELISA. It shows the amount of IL-8 measured in ng/ml in the range of 0 to 50 for each of the individual donors appearing along the X-axis.

### SUMMARY

These studies illustrate that IL-8 plays a significant role in the chemotactic activity of neutrophils found in sputum specimens from COPD patients. The lack of correlation with actual protein levels, as measured by ELISA, suggests inhibitory factor(s) in sputum that interfere with the ELISA detection.

TABLE 1

| | | | % | | % | | | | | | | | |
|---|---|---|---|---|---|---|---|---|---|---|---|---|---|
| | | | Inhibi- | | Inhibi- | | | | | | | | |
| Patient | | IL-8 | tion | | tion | | | | | | Anti- | | |
| # | Disease | (ng/ml) | 1:10 | Cl | 1:100 | Cl | Pulmonary | Steroids | 5-LO | COX | histamine | other | |
| 27 | COPD/CB | 2.72 | 100 | 2.4 | 100 | 1.6 | combivent | flovent | | | | | |
| | | | | | | | serevent | | | | | | |
| | | | | | | | albuterol | | | | | | |
| 14 | COPD | 0.32 | 82 | 4.17 | 100 | 1.56 | proventil | flovent | | aspirin | | hycosamine | |
| | | | | | | | atrovent | | | | | | |
| | | | | | | | serevent | | | | | | |
| 26 | CANCER | 33.09 | 89 | 3.3 | 93 | 1.9 | | | | | | | |
| 25 | COPD/ | 35.77 | 68 | 3.9 | 93 | 2 | albuterol | predinsone | | | | | |
| | ASTHMA | | | | | | serevent | flovent | | | | | |
| 41 | COPD | 2.64 | 56 | 4 | 88 | 1.6 | combivent | | | | | | |
| 35 | COPD/ | 0.685 | 52 | 2.4 | 84 | 1.6 | combivent | flovent | | naproxin | | | |
| | CANCER | | | | | | | | | | | | |
| 46 | COPD/CAD/ | 0.157 | 74 | 2.7 | 83 | 1.4 | combivent | prednisone | | vioxx | | prinvil | hytrin |
| | BPH/HBP/ | | | | | | | azmacort | | | | plavix | imdu |
| | GLACOMA | | | | | | | | | | | aciphex | |
| 22 | COPD/ | 12.89 | 54 | 4.9 | 81 | 2.9 | combivent | flovent | | | | synthroid | |
| | LUNG | | | | | | | | | | | prilosec | |
| | CANCER | | | | | | | | | | | | |
| 15 | COPD/HY- | 11.5 | 14 | 4.6 | 78 | 1.43 | theophylline | | | aspirin | | synthoid | |
| | POTHY- | | | | | | albuterol | | | | | | |
| | ROID | | | | | | atrovent | | | | | | |
| 38 | COPD/ | 0.15 | 60 | 3.2 | 77 | 1.9 | atrovent | prednisone | | | | lipitor | |
| | GERD/HY- | | | | | | albuterol | beclovent | | | | | |
| | PERCHOL- | | | | | | | | | | | | |
| | EST | | | | | | | | | | | | |
| 51 | COPD | 0.043 | 57 | 2.9 | 75 | 1.7 | theo-dur | | | | | fosamax | |
| | | | | | | | atrovent | | | | | premarin | |
| | | | | | | | albuterol | | | | | prevacid | |
| 34 | COPD/MI | 31.36 | 63 | 4.9 | 73 | 2.6 | | | | | | | |
| 45 | COPD | 0.774 | 48 | 3.2 | 68 | 1.47 | atrovent | | | | | premarin | |
| | | | | | | | uniphyll | | | | | zantac | |
| 13 | COPD | 11.07 | 66 | 4.3 | 62.2 | 2.6 | serevent | flovent | accolate | | | | |
| | | | | | | | albuterol | | | | | | |
| 50 | COPD | 0.177 | 55 | 6.5 | 60 | 2.6 | proventil | vancenase | | aspirin | | mycelex | |
| | | | | | | | atrovent | flovent | | | | calcium | |
| | | | | | | | serevent | | | | | | |
| 17 | EMPHY- | 44.3 | 47 | 4.3 | 56 | 2.4 | albuterol | | | aspirin | | | |
| | SEMA/ | | | | | | | | | | | | |
| | CANCER | | | | | | | | | | | | |
| 31 | COPD/ | 6.8 | 39 | 4.3 | 57 | 2 | albuterol | vanceril | | | | pepcid | |
| | GERD | | | | | | serevent | | | | | | |
| | | | | | | | atrovent | | | | | | |
| 29 | COPD | 2.94 | 43 | 4 | 50 | 2.3 | albuterol | flovent | | | claritan | | |
| | | | | | | | atrovent | flonase | | | | | |
| 24 | COPD | 1 | 26 | 4.26 | 44 | 2.3 | combivent | flovent | | | | | |
| | | | | | | | serevent | | | | | | |
| 44 | COPD/CAD | 22 | 28 | 3.3 | 36 | 1.7 | albuterol | | | aspirin | | tylox | lipito |
| | | | | | | | atrovent | | | | | prilosec | |
| 19 | COPD | 0.31 | 42 | 4.13 | 24 | 2.17 | atrovent | flovent | singular | aspirin | | synthroid | |
| | | | | | | | albuterol | | | | | atenlolol | |
| 16 | COPD | 0.82 | 0 | 3.25 | 17 | 1.9 | albuterol | vancenase | | aspirin | allegra | | |
| | | | | | | | serevent | | | | | | |
| | | | | | | | atrovent | | | | | | |
| | | | | | | | theophylline | | | | | | |
| 28 | COPD | 10.4 | 16 | 4.2 | 15 | 2 | serevent | flovent | | | | | |
| | | | | | | | combivent | | | | | | |
| | | | | | | | theo-dur | | | | | | |
| 18 | COPD? | 0.812 | 0 | 4.13 | 0 | 2.21 | albuterol | flovent | | | | prilosec | |
| | | | | | | | | | | | | notripylline | |
| 47 | COPD | 42.25 | 20 | 4.4 | 0 | 1.9 | atrovent | beclovent | | aspirin | | zantac | |
| | | | | | | | proventil | | | | | | |
| | | | | | | | tessalon | | | | | | |
| 42 | COPD | 2.6 | 5 | 2.75 | 0 | 1.5 | cxombivent | prednisone | singular | | | | |
| | | | | | | | serevent | azmacort | | | | | |
| 30 | COPD | 3.24 | 0 | 2.9 | 0 | 1 | | | | | | | |
| 33 | COPD/HY- | 14.84 | 0 | 2.56 | 0 | 1.86 | albuterol | prednisone | | | | allopurinol | |
| | PERTEN- | | | | | | atrovent | | | | | | |
| | SION/CAD | | | | | | | | | | | | |
| GERD | gastric eso- | | | | | | | | | | | | |
| | phygeal re- | | | | | | | | | | | | |
| | flux disease | | | | | | | | | | | | |
| CAD | coronary | | | | | | | | | | | | |
| | heart disease | | | | | | | | | | | | |

### Example 2

### Inhibition of lung inflammation by in vivo administration of IL-8 Antibodies

Methods:

Before evaluating the efficacy of using the human anti-IL-8 antibody ABX-IL8 in a rat model of IL-8-induced lung inflammation, an ex vivo study was used to determine whether human IL-8 can activate rat neutrophils, and whether the activation can be inhibited by the ABX-IL8 antibody.

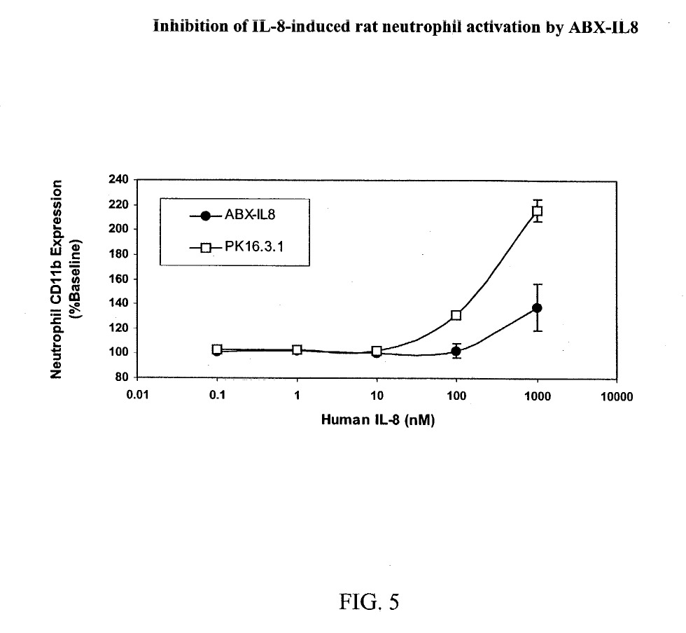

Five rats received human IgG2 control antibody PK16.3 or ABX-IL8 (0.3 or 3 mg/kg) intravenously. Twenty-four hours after administration, animals were bled. Whole blood neutrophil CD11b (a cell surface adhesion molecule and an activation marker for neutrophils) upregulation in response to human IL-8 (0.1-1000 nM) was evaluated by flow cytometry and the degree of inhibition (i.e. curve shift to right in ABX-IL8 treated animals) relative to control antibody. FIG. 5 shows the neutrophil CD11b expression (% baseline ranging from 80 to 240) as a function of the concentration of human IL-8 (ranging from 0.01 to 10000 nM). As shown in FIG. 5, IL-8 was indeed able to stimulate rat neutrophil activation, and ABX-IL8 was capable of inhibiting the human IL-8-induced rat neutrophil activation.

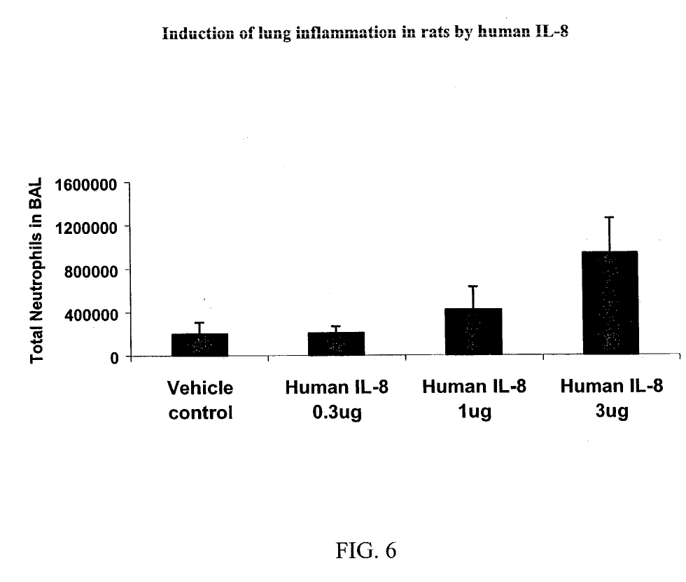

To evaluate the potential utility of systemic administration of ABX-IL8 as a treatment for COPD, a rat model of IL-8 induced lung inflammation was established by intratracheal (i.t.) administration of human IL-8. Eight rats received the vehicle control (PBS+0.1% low endotoxin bovine serum albumin), 0.3 μg of human IL-8, 1 μg of human IL-8, and 3 μg of human IL-8 intratrachealy. Four hours post i.t. instillation, bronchoalveolar lavage (BAL) was performed using 3×5 mL aliquots of saline. BAL fluid was analyzed for total and differential while blood cell counts. FIG. 6 shows the total count of neutrophils in the BAL. Intratracheal administration of human IL-8 (0.3, 1, and 3 μg) triggered dose dependent neutrophil migration into the airways of rats even though rats do not express IL-8. The largest total neutrophil count appeared in the rats receiving the 3 μg dose. Based on these results, a dose of 3 μg of human IL-8 was selected for the ABX-IL8 study because this dosage resulted in the highest level of neutrophil migration into the lungs.

To determine the effect of ABX-IL8 antibodies on IL-8 mediated neutrophil infiltration, Group 1 (ten rats) & Group 2 (nine rats) animals received no systemic treatment while Group 3 (eleven rats) animals received an i.v. dose of ABX-IL8 (5 mg/kg) on Days −4 and −1. Group 4 (eleven rats) animals received a dose of isotype matched control monoclonal antibody (PK16.3.1) (5 mg/kg) on Days −4 and −1. At Day 0, Group 1 rats received i.t. administered vehicle control (100 μL) and Groups 2, 3, and 4 rats received 3 μg human IL-8 i.t. in a volume of 100 μL.

Results:

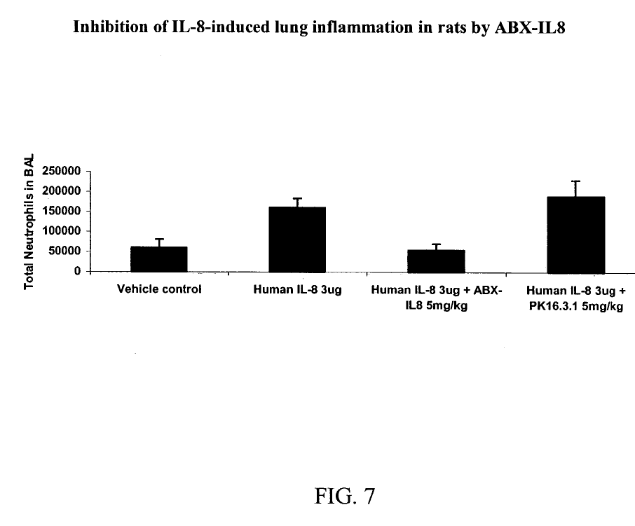

FIG. 7 shows the total neutrophil counts in the BAL of rats receiving the vehicle control, 3 μg of human IL-8, 3 μg of human IL-8+5 mg/kg of ABX-IL8, and 3 μg of human IL-8+5 mg/kg of control antibody PK16.3.1. As shown in FIG. 7, human IL-8 instilled i.t. triggered a 3-fold increase in neutrophil infiltration into the airways demonstrating that rat neutrophils can respond to human IL-8 in vivo. Intravenous administration of 5 mg/kg of ABX-IL8 resulted in significant inhibition of IL-8-induced airway neutrophil migration and accumulation (p<0.001) indicating that systemic exposure to ABX-IL8 can neutralize airway IL-8 and inhibit lung and airway inflammation.

### Example 3

### Assessment of Safety and Efficacy of ABX-IL8 in COPD Patients

Abstract:

This was a double blind, parallel-group, 3-month study in patients with COPD. Patients had evidence of obstructive pulmonary disease and had mild to moderately-severe disease defined by baseline forced expiratory volume in one second (FEV₁)≦70% of predicted and ≧30% of predicted. Patients also had a clinical diagnosis of chronic bronchitis. Patients with evidence of emphysema were included provided they also had symptoms consistent with chronic bronchitis. All subjects were >50 years of age and had a >20 pack-year history of smoking.

Patients enrolled in this study were randomized 1:1 to receive either ABX-IL8 (800 mg loading dose followed by two 400 mg treatment doses administered monthly) or placebo. The randomization is stratified by the baseline FEV₁, <40% or ≧40% of predicted. Furthermore, patients were stratified by the presence or absence of a bronchodilator response. A bronchodilator response was defined as ≧12% and ≧200 mL improvement in FEV₁ 30 minutes after inhaled albuterol.

Patients received three intravenous infusions over a period of 2 months (one 800 mg infusion at Month 0, one 400 mg infusion at Month 1 and one 400 mg infusion at Month 2). The study medication were infused via an infusion pump over 30-60 minutes.

The primary objective of this study was to demonstrate superior clinical efficacy for ABX-IL8 (loading dose of 800 mg followed by 400 mg administered every month for a total of three doses) compared with placebo, for the treatment of COPD over a 3-month period as assessed by the Transitional Dyspnea Index at Month 3

Secondary objectives of this study were 1) to demonstrate the safety and tolerability of ABX-IL8 in patients with chronic bronchitis; 2) to assess the effects of ABX-IL8 on patient-reported dyspnea as assessed by the UCSD Shortness of Breath Questionnaire; 3) to assess the effects of ABX-IL8 on exercise tolerance as measured by the 6 minute walk and modified Borg dyspnea scale; 4) to assess the effects of ABX-IL8 on health-related quality of life as assessed by the St. George's Respiratory Questionnaire; 5) to assess the effects of ABX-IL8 on rescue bronchodilator therapy as measured by a patient diary; 6) to assess the effects of ABX-IL8 on the incidence of COPD exacerbations and the time to first COPD exacerbation, 7) to assess the pharmacokinetics of ABX-IL8 dosed monthly in patients with COPD; 8) to assess the effects of ABX-IL8 on BAL fluid cell counts, IL-8 and other inflammatory mediator levels and to assess the level of ABX-IL8 in BAL fluid in the subset of patients undergoing bronchoscopy; 9) to assess the duration of action of ABX-IL8 by measuring spirometry, dyspnea, and St. George's Respiratory Questionnaire at study Months 4 & 5.

Methods:

The study began with 119 subjects, with 60 receiving the placebo and 59 receiving ABX-IL8 antibodies. These candidates were selected for participation in the study as described infra. Ultimately, 53 placebo subjects and 56 ABX-IL8 subjects completed the study. Reasons for subjects not completing the study included adverse events, lack of efficacy, or the subject's withdrawal of consent.

The subjects receiving the antibodies received an initial 800 mg loading dose and two subsequent 400 mg doses monthly; placebo subjects received placebo injections on the same schedule. Evaluations of the subjects were made at the baseline, at Week 2, and at Months 1-5.

The study sample size had an overall 80% power at an alpha level of 0.05 to detect a 150 mL difference in the improvement in FEV₁ at Month 3 compared to baseline in the patients treated with ABX-IL8 compared to placebo assuming the placebo patients demonstrate a 50 mL improvement and ABX-IL8 treated patients demonstrate a 200 mL improvement in FEV₁ with a common standard deviation of 265 mL.

Patients were stratified into four strata according to the patient's baseline FEV₁ (as a percent of predicted) and the magnitude of the patient's FEV₁ response to a bronchodilator at screening. The four strata were defined by:

1. FEV₁≧40% of predicted and <12% or <200 mL improvement in post-bronchodilator FEV₁,

2. FEV₁≧40% of predicted and 12% and ≧200 mL improvement in post-bronchodilator FEV₁,

3. FEV₁<40% of predicted and <12% or <200 mL improvement in post-bronchodilator FEV₁, and

4. FEV₁<40% of predicted and 12% and ≧200 mL improvement in post-bronchodilator FEV₁.

Within each stratum patients are randomized to one of two treatment groups (ABX-IL8 or placebo) at a ratio of 1:1.

The following inclusion and exclusion criteria were used to determine whether candidates would be appropriate subects for this study:

1. Inclusion Criteria

a. Patient is ≧50 years of age

b. Patient must have ≧20 pack-year history of smoking

c. Female patients who are post menopausal (Postmenopausal is defined as no menses for the previous 1 year. If cessation of menses is within 12 months, FSH must be documented as elevated into the postmenopausal range prestudy), surgically sterilized, or have a medical condition that prevents pregnancy (e.g., polycystic ovary disease) or are using an oral or implanted contraceptive, or an IUD and have a negative serum pregnancy test upon entry into this study or male partners willing to use double barrier birth control upon enrollment into this study. Female patients whose sole partner has had a vasectomy do not have to use birth control. All patients of child-bearing potential will continue to use an acceptable birth control method for the duration of the study or at least 5 months after the last dose of study drug whichever is longer.

d. Patient must have a clinical diagnosis of chronic bronchitis.

e. Excepting COPD, patient is judged to be in otherwise general good health based on medical history, physical examination, and routine laboratory screening tests.

f. Patient understands the study procedures and agrees to participate in the study by giving written informed consent.

g. Patient has a baseline severity of breathlessness of grade 1 or higher on the modified Medical Research Council dyspnea scale.

h. At sites performing bronchoscopy with BAL, patient is judged medically stable for the procedures, must have a pre-bronchodilator FEV₁≧40% of predicted and ≧1.5 liters, must have a room air arterial pCO₂<50 mmHg and pO2>60 mmHg and must provide written informed consent for the procedures.

i. Patient must successfully complete 6 minute walk at screening.

j. Patients must also meet the following criteria at the prestudy visit:

i. FEV₁≧30% of predicted and ≦70% of predicted

ii. FEV₁/FVC<70%

2. Exclusion Criteria

a. Patient has a concurrent medical/pulmonary disease that could confound or interfere with evaluation of efficacy including, but not limited to: bronchiectasis, cystic fibrosis, tuberculosis, asthma, α₁ antitrypsin deficiency or left-sided congestive heart failure.

b. Patient has a history of vasculitis.

c. Patient demonstrates a significant response to bronchodilators defined as >30% or >300 mL, whichever is greater, improvement in FEV₁ 30 minutes following inhaled albuterol treatment (180 μg) or has post-bronchodilator FEV₁>70% of predicted.

d. Patient requires oxygen therapy (other than nocturnal use) or will require oxygen therapy during exercise testing (6 minute walk).

e. Patient is mentally or legally incapacitated, has significant emotional problems at the time of the study, or has a history of psychosis.

f. Patient has angina with symptoms that occur at rest or minimal activity, and/or has a history of myocardial infarction, coronary angioplasty, or coronary arterial bypass grafting within the past 6 months.

g. Patient has a history of exercise related syncope or claudication.

h. Patient has uncontrolled hypertension [Note: patients with medically controlled hypertension (diastolic blood pressure ≦90, systolic blood pressure ≦150) may participate.]

i. Patient is seropositive for HIV.

j. Patient is positive for Hepatitis B surface antigen or Hepatitis C antibody (if patient is Hepatitis C antibody positive, AND the patient tests negative for Hepatitis C RNA, and patient is acceptable).

k. Patient has a history of neoplastic disease and does not meet one of the exceptions listed below. Patients with a history of leukemia, lymphoma, or myeloproliferative disease are ineligible for the study regardless of the time since treatment, and in such cases, no exceptions will apply.

Exceptions

i. Patients with adequately treated basal cell carcinoma, dermal squamous cell carcinoma or carcinoma in situ of the cervix.

ii. Patients with other malignancies which have been successfully treated ≧5 years prior to screening, where in the judgment of both the investigator and treating physician, appropriate follow-up has revealed no evidence of recurrence from the time of treatment through the time of screening.

iii. Patients who, in the joint opinion of the Abgenix monitor and investigator, are highly unlikely to sustain a recurrence during the duration of the study.

l. Patient has a history of any illness that, in the opinion of the investigator, might confound the results of the study or pose additional risk to the patient.

m. In the opinion of the investigator or the medical monitor, patient has clinically significant abnormalities on prestudy clinical examination or laboratory safety tests.

n. Patient is currently a user (including “recreational use”) of any illicit drugs, or has a history (within the past 5 years) of drug or alcohol abuse.

o. Patient has donated a unit of blood or plasma, or participated in another clinical study with an investigational agent within the last 4 weeks. (Patients unwilling to refrain from donation of blood or blood products while participating in the protocol will also be excluded.)

p. Patient has previously been exposed to ABX-IL8 in a clinical study.

q. History of the following specific laboratory abnormalities at screening:

Leukopenia (<3×10⁹/L)

Neutropenia (<1.5×10⁹/L)

Anemia (Hgb<11 g/dL)

Thrombocytopenia (<100×10⁹/L)

Elevated serum creatinine (>1.5 mg/dL)

Transaminases (ALT or AST) greater than two times the upper limit of normal

PT>15 seconds or PTT>40 seconds

r. Recent history (within 2 months of study Visit 2) of COPD exacerbation or pneumonia requiring hospitalization or emergency room treatment

s. History of infection (within 2 weeks of study start) requiring hospitalization or intravenous antibiotics and/or clinical signs/symptoms of active infection.

t. Recent surgery (within 1 month of study start).

u. Surgical or non-surgical wounds that are currently healing.

3. Previous or Concurrent Medication

a. Patients may not be receiving nor have discontinued oral or parenteral corticosteroids within one month prior to Study Visit 2 (first dose of study medication).

b. Patients may not be receiving nor have discontinued inhaled corticosteroids, leukotriene receptor antagonists, theophylline-containing preparations or oral β agonists within one week prior to Study Visit 2 (first dose of study medication).

c. Patients may not be receiving oral or parenteral antibiotics at screening or at study start.

d. Patients may use inhaled long-acting β agonists (salmeterol xinafoate) but must refrain from their use for at least 12 hours prior to each study visit.

e. Patients may use inhaled ipatropium bromide but must refrain from their use for at least 6 hours prior to each study visit.

f. Patients may use inhaled short-acting β agonists (e.g. albuterol) but must refrain from their use at least 6 hours prior to each study visit. The use of inhaled bronchodilators administered on an ‘as needed’ basis will be recorded in the patient's diary during the course of the study.

g. Patients may not be receiving warfarin or heparin containing compounds at screening or during the treatment period.

Study Visits:

Study Visits were conducted according to the following protocols:

Study Visit 1

Potential patients were evaluated to determine whether they fulfilled the entry requirements. Investigators performed a physical examination, spirometry (pre and post bronchodilator), modified Medical Research Council dyspnea scale, 6 minute walk and screening laboratories. Patients who successfully completed screening were eligible for randomization. For these patients, Study visit 2 was scheduled two weeks after study visit 1.

Study Visit 2

Study visit 2 was the baseline visit. The following procedures were performed:

1. Patients completed the St. George's Respiratory Questionnaire and UCSD Shortness of Breath Questionnaire

2. Baseline Dyspnea Questionnaire was administered

3. Vital signs and weight

4. Abbreviated physical examination

5. Spirometry, lung volumes and diffusing capacity

6. Spirometry 30 minutes post 180 μg inhaled albuterol

7. 6 minute walk with Borg Dyspnea Scale

8. Bronchoscopy with BAL was performed in a subset of patients at the designated BAL sites

9. Urine pregnancy test

10. Urinalysis

11. Blood was drawn for the following before dosing:

CBC with differential and absolute platelet count

Serum chemistry

HAHA

Trough ABX-IL8 PK

Endogenous free serum IL-8 levels; serum was archived for cytokine analyses.

Study drug was administered intravenously under sterile conditions over a period of approximately 30 minutes. Regular assessments of vital signs were obtained during and for 30 minutes following study drug infusion. Blood was drawn for peak ABX-IL8 PK 30 minutes after completion of dosing. Adverse events were recorded during and following study drug infusion. Patients were given a diary to log their β agonist use.

Study Visit 3

Study Visit 3 occured 1 month following Study Visit 2. The following procedures were performed:

1. Recording of patients β agonist rescue medication

2. Recording of adverse events

3. Patients completed the St. George's Respiratory Questionnaire and UCSD Shortness of Breath Questionnaire

4. Transitional Dyspnea Index Questionnaire was administered

5. Vital signs and weight

6. Physical examination

7. Spirometry (pre and post bronchodilator)

8. 6 minute walk with Borg Dyspnea Scale

9. Urine pregnancy test

10. Urinalysis

11. Blood was drawn for the following before dosing:

CBC with differential and absolute platelet count

Serum chemistry

Trough ABX-IL8 PK

Endogenous free serum IL-8 levels; serum was archived for other cytokine analyses

Study drug was administered intravenously under sterile conditions over a period of approximately 30 minutes. Regular assessments of vital signs was obtained during and for 30 minutes following study drug infusion. Blood was drawn for peak ABX-IL8 PK (approximately 30 minutes after completion of dosing).

Study Visit 4

Study Visit 4 occured approximately 2 months after Study Visit 2. The following procedures were performed:

1. Recording of patients β agonist rescue medication

2. Recording of adverse events

3. Patients completed the St. George's Respiratory Questionnaire and UCSD Shortness of Breath Questionnaire

4. Transitional Dyspnea Index Questionnaire was administered

5. Vital signs and weight

6. Physical examination

7. Spirometry (pre and post bronchodilator)

8. 6 minute walk with Borg Dyspnea Scale

9. Urine pregnancy test

10. Urinalysis

11. Blood was drawn for the following before dosing:

CBC with differential and absolute platelet count

Serum chemistry

Trough ABX-IL8 PK

Endogenous free serum IL-8 levels; serum was archived for other cytokine analyses

Study drug was administered intravenously under sterile conditions over a period of approximately 30 minutes. Regular assessments of vital signs was obtained during and for 30 minutes following study drug infusion. Blood was drawn for peak ABX-IL8 PK (approximately 30 minutes after completion of dosing).

Study Visit 5

Study Visit 5 occured approximately 3 months after Study Visit 2. The following procedures were performed:

1. Recording of patients β agonist rescue medication

2. Recording of adverse events

3. Patients completed the St. George's Respiratory Questionnaire and UCSD Shortness of Breath Questionnaire

4. Transitional Dyspnea Index Questionnaire was administered

5. Vital signs and weight

6. Physical examination

7. Spirometry, lung volumes and diffusing capacity

8. Spirometry 30 minutes post bronchodilator

9. 6 minute walk with Borg Dyspnea Scale

10. Bronchoscopy with BAL was performed in a subset of patients at the designated BAL sites

11. Urine pregnancy test

12. Urinalysis

13. Blood was drawn for the following:

CBC with differential and absolute platelet count

Serum chemistry

HAHA

Trough ABX-IL8 PK

Endogenous free serum IL-8 levels; serum was archived for other cytokine analyses

Study Visit 6—Safety Follow-up

Study Visit 6 occured approximately 4 months after Study Visit 2. The following procedures were performed:

1. Recording of adverse events and concomitant medications

2. Patients completed the St. George's Respiratory Questionnaire and UCSD Shortness of Breath Questionnaire

3. Transitional Dyspnea Index Questionnaire was administered

4. Vital signs and weight

5. Physical examination

6. Spirometry (pre and post bronchodilator)

7. Blood was drawn for the following:

CBC with differential and absolute platelet count

Serum chemistry

Trough ABX-IL8 PK

HAHA

Serum was archived for cytokine analyses

Study Visit 7—Safety Follow-up

Study Visit 7 occured approximately 5 months after Study Visit 2. The following procedures were performed:

1. Recording of adverse events and concomitant medications

2. Patients completed the St. George's Respiratory Questionnaire and UCSD Shortness of Breath Questionnaire

3. Transitional Dyspnea Index Questionnaire was administered

4. Vital signs and weight

5. Physical examination

6. Spirometry (pre and post bronchodilator)

7. Blood was drawn for the following:

CBC with differential and absolute platelet count

Serum chemistry

Trough ABX-IL8 PK

HAHA

Serum was archived for cytokine analyses

The procedures performed at each study visit are shown in Table 2.

TABLE 2
Study Procedures

| Study Visit | 1 | 2 | 3 | 4 | 5 | 6 | D/C | 7 | 8 | |
|---|---|---|---|---|---|---|---|---|---|---|
| Month | | 0 | | 1 | 2 | 3 | | FU1 | FU2 | 7 |
| Week | | | 2 | | | | | | | |
| Study Period | S | T | T | T | T | T | | F | F | |
| Informed Consent | X | | | | | | | | | |
| Eligibility | X | | | | | | | | | |
| Medical and Medication History | X | | | | | | | | | |
| Smoking History and status | X | X | X | X | X | X | X | X | X | |
| Concomitant Medication | | X | X | X | X | X | X | X | X | |
| Adverse Events including Serious | | X | X | X | X | X | X | X | X | |
| Complete Physical Exam Examination | X | | | | | | | | | |
| Abbreviated Physical Exam | | X | X | X | X | X | X | X | X | |
| Vital Signs | X | X | X | X | X | X | X | X | X | |
| ECG/CXR | X | | | | | | | | | |
| Modified Medical Research Council | X | | | | | | | | | |
| St. George's Respiratory Questionaire | | X | | X | X | X | X | X | X | |
| UCSD Shortness of Breath | | X | X | X | X | X | X | X | X | |
| Baseline Dyspnea Index | | X | | | | | | | | |
| Transitional Dyspnea Index | | | X | X | X | X | X | X | X | |
| Spirometry (pre and post | X | X | X | X | X | X | X | X | X | |
| bronchodilator) | | | | | | | | | | |
| Lung Volumes & Diffusing Capacity | | X | | | | X | X^{f} | | | |
| 6 min Walk with Modified Borg Dyspnea | X | X | | X | X | X | X | | | |
| Scale | | | | | | | | | | |
| Record Rescue Therapy use from | | X | X | X | X | X | X | X | X | |
| Patient Diary | | | | | | | | | | |
| Bronchoscopy with endobronchial | | X | | | | X | X^{g} | | | |
| biopsies and BAL^{a} | | | | | | | | | | |
| Study Medication Administration | | X | | X | X | | | | | |
| 5 Month Follow-up for Pregnancy^{d} | | | | | | | | | | X |
| CBC/Diff and Platelet Count | X | X | X | X | X | X | X | X | X | |
| Serum Chemistry | X | X | X | X | X | X | X | X | X | |
| Room Air Arterial Blood Gas^{a} | X | | | | | | | | | |
| PT/PTT | X | | | | | | | | | |
| HIV and Hepatitis Screen | X | | | | | | | | | |
| HAHA | | X | | | | X | X | X | X | |
| PK^{b} | | X | | X | X | X | X | X | X | |
| Serum Pregnancy Test^{c} | X | | | | | | | | | |
| Urine Pregnancy Test^{d} | | X | | X | X | X | X | X | X | |
| Urinalysis | X | X | X | X | X | X | X | | | |
| Serum for IL-8 and other inflammatory | | X | | X | X | X | X | X | X | |
| markers | | | | | | | | | | |
| Whole Blood for DNA Archive^{e} | | X | | | | | | | | |

S, T, F, D S = Screening Visit, T = Treatment Period Visits; F = Follow Up Visits; D = Discontinuation Visit.
X Required procedures.
^{a}Room air Arterial Blood Gas and bronchoscopy with BAL will be performed on a subset of patients at designated bronchoscopy sites, only.
^{b}Two pharmacokinetic samples will be drawn at all visits where patients are dosed (one prior to dosing and one approximately 30 minutes after completion of dosing). One pharmacokinetic sample will be drawn at visits in which patients are not dosed.
^{c}To be performed on female patients, only
^{d}To be performed on female patients of child bearing potential, only.
^{e}Serum will be archived for ctyokine analyses.

Results

It was discovered that the Mean TDI Score for subjects receiving the ABX-IL8 antibodies improved relative to the Mean TDI Score for subjects receiving the placebo. It was also discovered that there was a favorable change in the FEV1 in subjects receiving the antibody treatment who had a Baseline Dyspnea Index (BDI) of =>7, compared to the placebo subjects having the same BDI. Thus, the treatment of COPD with antibodies against IL-8 was effective to reduce the effects of COPD in the patients.

### Example 4

### Treatment of COPD in Humans

A patient suffering from COPD is identified. A dosage of 5 mg/kg of the ABX-IL8 antibody is administered by intravenous injection to the patient. A booster administration is given three weeks later, and every three weeks thereafter. The ABX-IL8 antibody causes a partial or complete inhibition of neutrophil chemotaxis in the inflamed respiratory tissues. This inhibition of neutrophil chemotaxis reduces the severity of tissue damage to the lungs and air passages caused by the patient's immune response.

### Example 5

### Treatment of Chronic Bronchitis in Humans

A patient suffering from COPD characterized by chronic bronchitis is identified. A dosage of 5 mg/kg of the ABX-IL8 antibody is administered by intravenous injection to the patient. A booster administration is given three weeks later, and every three weeks thereafter. The ABX-IL8 antibody causes a partial or complete inhibition of neutrophil chemotaxis in the inflamed respiratory tissues. This inhibition of neutrophil chemotaxis reduces the severity of tissue damage to the lungs and air passages caused by the patient's immune response.

### Example 6

### Treatment of Emphysema in Humans

A patient suffering from COPD characterized by emphysema is identified. A dosage of 5 mg/kg of the ABX-IL8 antibody is administered by intravenous injection to the patient. A booster administration is given three weeks later, and every three weeks thereafter. The ABX-IL8 antibody causes a partial or complete inhibition of neutrophil chemotaxis in the inflamed respiratory tissues. This inhibition of neutrophil chemotaxis reduces the severity of tissue damage to the lungs and air passages caused by the patient's immune response.

### Example 7

### Treatment of Irreversible Asthma in Humans

A patient suffering from COPD characterized by late-stage or irreversible asthma is identified. A dosage of 5 mg/kg of the ABX-IL8 antibody is administered by intravenous injection to the patient. A booster administration is given three weeks later, and every three weeks thereafter. The ABX-IL8 antibody causes a partial or complete inhibition of neutrophil chemotaxis in the inflamed respiratory tissues. This inhibition of neutrophil chemotaxis reduces the severity of tissue damage to the lungs and air passages caused by the patient's immune response.

What is claimed is:

1. A method of treating a patient suffering from symptoms of Chronic Obstructive Pulmonary Disease (COPD) comprising administering an amount of an antibody specific for human interleukin-8 effective to reduce said symptoms.

2. The method of claim 1, wherein the antibody neutralizes said human interleukin-8 in the patient.

3. The method of claim 1, wherein the antibody down-regulates the activity of interleukin-8 in the patient.

4. The method of claim 1, wherein the antibody is administered by one or more of the routes selected from the group consisting of intravenous, intraperitoneal, inhalation, intramuscular, subcutaneous and oral.

5. The method of claim 1, wherein the antibody is a monoclonal antibody.

6. The method of claim 1, wherein the antibody is a fully human antibody.

7. The method of claim 6, wherein the fully human antibody is an ABX-IL8 antibody.

8. A method of treating a patient suffering from symptoms of chronic bronchitis comprising administering an amount of an antibody specific for interleukin-8 effective to reduce said symptoms.

9. The method of claim 8, wherein the antibody neutralizes said human interleukin-8 in the patient.

10. The method of claim 8, wherein the antibody down-regulates the activity of interleukin-8 in the patient.

11. The method of claim 8, wherein the antibody is administered by one or more of the routes selected from the group consisting of intravenous, intraperitoneal, inhalation, intramuscular, subcutaneous and oral.

12. The method of claim 8, wherein the antibody is a monoclonal antibody.

13. The method of claim 8, wherein said antibody is a fully human antibody.

14. The method of claim 13, wherein the fully human antibody is an ABX-IL8 antibody.

15. A method of treating Chronic Obstructive Pulmonary Disease (COPD) in a human subject, comprising the step of administering to said subject a therapeutically effective amount of an antibody specific for interleukin-8, formulated in a pharmaceutically acceptable vehicle.

16. The method of claim 15, wherein the antibody neutralizes said human interleukin-8 in the patient.

17. The method of claim 15, wherein the antibody down-regulates the activity of interleukin-8 in the patient.

18. The method of claim 15, wherein the antibody is administered by one or more of the routes selected from the group consisting of intravenous, intraperitoneal, inhalation, intramuscular, subcutaneous and oral.

19. The method of claim 15, wherein the antibody is a monoclonal antibody.

20. The method of claim 15, wherein said antibody is a fully human antibody.

21. The method of claim 20, wherein the fully human antibody is an ABX-IL8 antibody.

22. The method of claim 15 wherein the pharmaceutically acceptable vehicle comprises phosphate buffered saline.
